(19) United States
(12) Patent Application Publication
(10) Pub. No.: US 2026/0284033 A1
(43) Pub. Date: Sep. 24, 2026

(54) COMBINATION THERAPY WITH LP-PLA2 INHIBITOR AND A SECOND AGENT

(71) Applicant: SciNeuro Therapeutics Inc., Rockville, MD (US)

(72) Inventors: Min LI, Rockville, MD (US); Guojun BU, Rockville, MD (US); Gang AN, Rockville, MD (US); Yuka MARTENS, Rockville, MD (US); Andreas ZEMBRZYCKI, Rockville, MD (US); Dai-Shi SU, Rockville, MD (US)

(73) Assignee: SciNeuro Therapeutics Inc., Rockville, MD (US)

(21) Appl. No.: 19/476,619

(22) PCT Filed: Apr. 17, 2024
(86) PCT No.: PCT/US2024/024865

§ 371 Date: Oct. 17, 2025

Related U.S. Application Data
(60) Provisional application No. 63/496,892, filed on Apr. 18, 2023.

(51) Int. Cl. A61K 31/4709; A61K 31/517; A61K 45/06

(52) U.S. Cl. CPC A61K 31/4709; A61K 31/517; A61K 45/06

(57) ABSTRACT

The invention provides combination therapy involving the inhibition of lipoprotein-associated phospholipase A2 (Lp-PLA2) and a second therapy. To facilitate the practice of the invention, the invention also provides a composition comprising a first agent comprising an Lp-PL2 inhibitory molecule, a second agent, and a pharmaceutically-acceptable carrier. In certain embodiments, the second agent comprises an amyloid-targeting molecule, an agent that lowers the level of amyloid in the brain or body, a tau-targeting molecule, a vasculature-targeting molecule, an inflammation-targeting molecule, and/or a molecule that modulates a mechanism of action that is orthogonal from Lp-PLA2 inhibition. Combinations of such second agents are contemplated as well. While the invention can be used as prophylaxis and treatment for a variety of disorders, in certain embodiments, the invention is employed against vascular damage or inflammation associated with a disease, condition, or therapy-related side effect, particularly in the context of neurological or neuromuscular conditions.

### CROSS-REFERENCED TO RELATED APPLICATIONS

This application claims priority to and the benefit of U.S. Patent Application 63/496,892, filed Apr. 18, 2023, the entire content of which is incorporated herein in its entirety.

### BACKGROUND OF THE INVENTION

Medical complications, conditions, and age-related diseases such as diabetes, neurodegenerative diseases (NDDs), neurological injuries or diseases, mental disorders, proteinopathies, and vascular diseases vary in type and scope. Many therapies, often monotherapies, have been approved and are under investigation for use in treatment or prophylaxis for an array of these conditions, diseases, and disorders. However, because such conditions, diseases, and disorders often are multifactorial, such therapies are not as safe and effective as would be desired, nor are they available to as broad a patient base as would be desired.

Neurodegenerative diseases (NDDs) such as Alzheimer's disease (AD), Parkinson's disease (PD), amyotrophic lateral sclerosis (ALS), frontotemporal dementia (FTD), and Huntington's disease (HD) are defined symptomatically and pathologically. For example, an AD diagnosis has typically been defined by clinical symptoms and the presence of amyloid plaques and tau tangles in postmortem brain samples. Recently, AD has been defined biologically as a disease with the presence of amyloid-β (Aβ) plaques and tau tangles, with or without clinical symptoms. The presence of Aβ plaques and tau tangles can be measured in living persons by an array of imaging and fluid biomarkers. While positron emission tomography (PET) imaging with specific tracers can detect brain Aβ amyloid and tau tangles directly, the levels of fluid biomarkers, such as Aβ42, Aβ40, total tau proteins or phosphorylated tau (pTau) species including but not limited to pTau181, pTau217, and the ratio of Aβ42/40 in blood, plasma, serum, and cerebrospinal fluid (CSF) can serve as surrogates of brain Aβ amyloid and tau tangles. Although neurodegeneration is often an accompanying change associated with AD development as measured by fludeoxyglucose (FDG) PET and magnetic resonance imaging (MRI), it is not a prerequisite of AD. As such, AD patients can be non-symptomatic (preclinical stage) or symptomatic (clinical stage).

Disease-modifying therapy to treat AD has not been available until recently. This is exemplified by anti-amyloid drugs such as Aducanamab and Lecanamab, which are typically employed as monotherapies. However, the safety of these agents is of significant concern, which has limited their therapeutic doses and potential greater efficacy. While these and other current therapies targeting amyloid proteins including anti-Aβ monoclonal antibodies hold promise for the disease-modifying treatment of AD by reducing Aβ levels in a dose-dependent manner, the overall efficacy in slowing cognitive decline is moderate, suggesting that additional pathogenic pathways independent of amyloids such as vascular damages and inflammation also drive cognitive decline. Further, anti-amyloid therapies often induce dose-dependent cerebral microbleeds and vascular damages as drug treatment-induced side effects. Such vascular damage and inflammation are heterogeneous and manifests in various forms including but not limited to amyloid related imaging abnormalities (ARIA), notably ARIA-E relating to vascular edema due to blood brain barrier (BBB) leakage and fluid built-up, and ARIA-H relating to cerebral micro or macro-hemorrhages caused by blood vessel rupture. Due to these treatment-induced side effects the therapeutic window of these agents is limited, hence, the efficacious dose is not optimal. Currently, when patients experience treatment-related vascular side-effects, the clinical response typically is to discontinue therapy, unfortunately denying the benefits of anti-amyloid therapy to such patients.

In addition to targeting amyloid directly, certain therapies are designed to restore the homeostasis or improve the phagocytic efficiency of glia cells in the brain, including astrocytes and microglia. For example, therapies targeting triggering receptor expressed on myeloid cells 2 (TREM2) have similarly been shown to reduce amyloid levels in the brain in preclinical animal models. In a clinical trial, the treatment with a TREM2-targeting monoclonal antibody also caused vascular damage consistent with ARIA described above.

Similarly, heterogeneous vascular damage and inflammation are independent disease pathologies of NDDs and are defined as vascular dementia or vascular cognitive impairment and dementia (VCID). Cerebral small vessel disease (CSVD) is a common and pathological, clinical, and radiological heterogenous cerebrovascular deterioration associated with VCID. The VCID leads to cognitive decline as a consequence of vasculature impairments and is subdivided into the four main categories as post-stroke dementia, subcortical ischemic vascular dementia, multi-infarct dementia, and mixed dementia. Since the biological mechanisms of VCID and CSVD are largely unrelated to AD pathologies, they cannot be addressed by molecules targeting amyloid and/or tau burden in the brain and body, again suggesting the need for improved therapies for these pathologies.

Many especially elderly patients, as well as patients with advanced stage AD, or subjects presenting with vascular co-morbidities or a history of cerebral hemorrhage and microbleeds currently have restricted or no access to potential disease-modifying and life-saving amyloid-targeting therapies, as they are contraindicated for such. For example, patients with cerebral amyloid angiopathy (CAA), a common disease affecting the brain blood vessels characterized by Aβ protein deposition in small and medium-sized leptomeningeal and cortical blood vessels, are at risk for ARIA; also, anti-amyloid antibody treatment-mediated rapid removal of parenchymal Aβ in CAA patients can induce significant inflammation and autoimmune reactions that further increase vascular and neuronal damage. In the case of cerebral amyloid angiopathy related inflammation (CAAri), the APOE4 genotype, an allele carried by certain individuals, is over-represented and increases the patient's risk for developing these types of pathologies and conditions.

Furthermore, the pathologic mechanisms that are driving neurodegenerative disease (NDD) onset, progression and severity often are multifold. Accordingly, the treatment of such polygenic and multifactorial diseases may require the normalization of more than one biological pathway. This is consistent with the low success rate of monotherapies in treating neurological diseases and exemplifies the urgent need to provide more potent approaches targeting different aspects of disease-driving pathways.

To address this urgent need, as discussed more fully below, the present invention pertains to combination therapies comprising an inhibitor of lipoprotein-associated phospholipase A2 (Lp-PLA2) and other agents to treat human disease.

Lp-PLA2 is a secreted enzyme that plays a critical role in the metabolism of lipoproteins. It is primarily expressed in the liver from where it is secreted into the bloodstream and often binds to low-density lipoprotein (LDL) particles. Catalytic processing of LDL particles by Lp-PLA2 in the bloodstream generates potent pro-inflammatory mediators promoting vascular defects, such as driving the pathogenesis of atherosclerosis. In preclinical atherosclerosis animal models, genetic deletion of Lp-PLA2 or inhibition of its enzymatic activity alleviated the development and progression of atherosclerotic lesions. In humans, elevated levels of Lp-PLA2 have been associated with an increased risk of cardiovascular events and pathology.

In the CNS, upregulation of Lp-PLA2, for example, caused by stroke, aggravates post-stroke ischemic lesions and tissue damage. In AD patients, it has been shown that Lp-PLA2 inhibition repairs vascular damages and slows cognitive decline.

Lp-PLA2 inhibitors have been developed for the treatment of cardiovascular diseases. The clinical trial results suggested that the Lp-PLA2 inhibitor, darapladib, may only be effective in patients with high levels of Lp-PLA2.

In BBB leakage animal models, the treatment with Lp-PLA2 inhibitors, such as darapladib, reduced BBB leakage and improved vascular dementia pathology. In a placebo-controlled, randomized phase 2 clinical study, the Lp-PLA2 inhibitor, rilapladib, was well tolerated, improved disease biomarkers, and importantly slowed down cognitive decline in AD patients.

Mechanistically, some preclinical data suggested that Lp-PLA2 inhibition could reduce amyloid levels, but this has not been replicated independently. Similarly, analyses of amyloid metabolism, turnover, and dynamics in CSF and plasma in the rilapladib phase 2 trial mentioned previously did not support an involvement of Lp-PLA2 in amyloid dynamics. Instead, it suggested the beneficial effect observed in rilapladib-treated AD patients were mediated by improving or preventing BBB leakage, breakdown, and/or vascular inflammation and damages. Therefore, Lp-PLA2 inhibition is a promising mechanism to treat and/or prevent diseases and disorders with a component of vascular damage, such as BBB leakage, and inflammation, for example, but not limited to nervous system diseases including NDDs, either on its own or in combination with another therapy.

### BRIEF SUMMARY OF THE INVENTION

The present invention provides such alternative therapies to effectively treat neurological, neuromuscular, and other diseases and disorders, including polygenic and multifactorial neurological diseases. The invention provides combination therapy involving the inhibition of lipoprotein-associated phospholipase A2 (Lp-PLA2) and a second therapy. While the invention can be used as prophylaxis and treatment for a variety of disorders, in certain embodiments, the method and use are employed therapeutically or prophylactically to prevent, improve, and/or treat vascular damage or inflammation, particularly in the context of neurological or neuromuscular conditions, such as those caused by aging, human diseases, and/or drug-treatments or a combination thereof.

In one embodiment, the invention provides a method of treatment comprising administering to a subject a first agent comprising an Lp-PLA2 inhibitory molecule, and the use of such first agent in connection with such administration, and administering a second agent to the subject, and the use of such second agent in connection with such administration, to achieve prophylaxis or therapeutic treatment of a disease state in the subject. Also, while the second agent for implementation in the context of the present invention can comprise many types of agents, in certain embodiments, the second agent comprises an amyloid-targeting molecule, an agent that lowers the level of amyloid in the brain or body, a tau-targeting molecule, vasculature-targeting molecule, an inflammation-targeting molecule, or a molecule that modulates a mechanism of action that is orthogonal from Lp-PLA2 inhibition. Combinations of second agents are contemplated as well.

In this respect, the invention provides an improved method of treatment or prophylaxis involving such second agents. The improvement lies in combining monotherapies, as described herein, with the first agent comprising an Lp-PLA2 inhibitory molecule. As a result of this improvement, the side-effects of such monotherapy agents (some of which are discussed above) can be mitigated. Also, as a result of the improvement provided by the present invention, the combination treatment can expand the clinically safe/approved dose range of a monotherapy agents or disease-modifying treatments. Co-treatment involving Lp-PLA2 inhibitor in accordance with the present invention can, for example, enable an increase of safety margin of a second agent. The consequence includes expanding a clinically safe/approved dose range of a second agent such as Aducanamab and Lecanamab as non-limiting examples, the safe and efficacious prolongation of a treatment regiment, compared to a mono-therapy alone, and expansion of indications to cover groups of patients otherwise unsuitable or excluded from such monotherapy.

Furthermore, to facilitate the inventive use and the practice of the inventive method, the invention also provides a composition comprising a first agent comprising an Lp-PLA2 inhibitory molecule, a second agent, and a pharmaceutically-acceptable carrier.

### DETAILED DESCRIPTION OF THE INVENTION

In one aspect, the invention provides a method for therapeutic or prophylactic treatment of diseases with a component of vascular damage and/or inflammation within a subject comprising administering a first agent to the subject, wherein the first agent comprises a lipoprotein-associated phospholipase A2 (Lp-PLA2)-inhibitor molecule, and administering a second agent to the subject, whereby the combined administration of the first and the second agent effects therapeutic prophylaxis or treatment of vascular damage and/or inflammation within the subject. In a related aspect, the invention provides the use of a first agent comprising a Lp-PLA2-inhibitor molecule and a second agent in the treatment or prophylaxis of vascular damage and/or inflammation within a subject.

In the context of the present invention, the method (or “use”) can be used prophylactically, such as to mitigate the possible vascular damage and/or inflammation that may be associated with the administration of the second agent or due to an underlying condition or risk factor present within the subject. The prophylactic effect need not be absolute; in the context of the present invention, it is of sufficient prophylactic effect if the risk to the subject of vascular damage and/or inflammation is reduced as a result of the inventive method or use. Also, in the context of the present invention, the method (or “use”) can be employed therapeutically to treat a patient already suffering from (e.g., symptomatic) or otherwise diagnosed (symptomatic or asymptomatic) with vascular damage and/or inflammation. The therapeutic effect need not be absolute; in the context of the present invention, it is of sufficient therapeutic effect if vascular damage and/or inflammation is reduced or mitigated as a result of the inventive method or use. In an embodiment, the Lp-PLA2 inhibitor is used to mediate reduction of vascular damage and/or inflammation to expand a clinically safe/approved dose range of a second agent or a disease-modifying treatment.

While any suitable agent able to inhibit Lp-PLA2 can be employed as the “first agent” in the context of the present invention, preferred agents include darapladib, rilapladib, GSK3206906A (SNP318), and a combination thereof, which are offered as non-limiting examples. Moreover, the “first agent” can be or comprise a fragment, derivative, modified format, or mimetic of darapladib, rilapladib, GSK3206906A (SNP318), and a combination thereof which retains the ability to inhibit Lp-PLA2. Additional non-limiting examples of Lp-PLA2-inihibiting agents that can be used as the “first agent” in the context of the present invention are described in, for example, International Patent Publications WO96/13484, WO96/19451, WO97/02242, WO97/217675, WO97/217676, WO97/41098, WO97/41099, WO99/24420, WO00/10980, WO00/66566, WO00/66567, WO00/68208, WO01/60805, WO01/60805, WO02/030904A1, WO02/030911A1, WO02/30904, WO02/30911, WO03/016287, WO03/041712, WO03/042179, WO03/042206, WO03/042218, WO03/086400, WO03/087088, WO05/021002, WO06/063791A1, WO06/063811A1, WO06/063812A1, WO06/063813A1, WO21/089032A1; US Patent Publications US2006/106017A1 and US2015/0183748; U.S. Pat. Nos. 6,649,619, 7,153,861, and 7,169,924, and U.S. Provisional Patent Applications 60/829,327 and 60/829,328, each of which is incorporated herein its entirety by reference. Additionally or alternatively, irreversible acylating inhibitors that can be used in embodiments of the present invention are described, for example, in Tew et al, Biochemistry, 37, 10087 (1998), which is incorporated herein its entirety by reference. Additionally or alternatively, eversible inhibitors that can be used in embodiments of the present invention are described, for example, in in US Patent Publication US2005/0033052A1 and in International Patent Publications WO02/30904, WO03/042218, WO03/042206, WO03/042179, WO03/041712, WO03/086400, and WO 03/87088, each of which is incorporated herein its entirety by reference. Additional or alternative Lp-PLA 2 inhibitors that can be used in embodiments of the present invention also include known agents, such as statins with Niacin and fenofibrate.

The invention is not limited to these agents presented as non-limiting examples above but can comprise the use of any agent able to inhibit Lp-PLA2 in the context of the present invention and be compatible with therapeutic use. Such can be a known agent or an agent discovered to be an Lp-PLA2-inhibitor. Typically, inhibition of Lp-PLA2, and hence the identification of a suitable first agent, involves an in vitro, enzymatic assay with colorimetric or radiometric detection of signal. However, any method suitable for identification an inhibitor of Lp-PLA2 can be employed.

In the context of the present invention, Lp-PLA2 inhibition can be assessed by any suitable method known to a person of ordinary skill in the art. For example, the inhibition can be assessed by assaying for the expression of Lp-PLA2 and/or the inhibition of protein activity. Moreover, complete inhibition is not necessary; the agent can achieve inhibition of Lp-PLA2, when assessed, at a level of at least about 20%, e.g., at least about 30%, at least about 40%, at least about 50%, at least about 60%, at least about 70%, at least about 80%, at least about 90%, at least about 95%, or at least about 98%. In this respect, for example, it has been reported that rilapladib can achieve approximately 80% inhibition of Lp-PLA2 when at a dosage of 250 mg to human subjects. However, other dosages also are contemplated in the context of the present invention.

For use in the context of the present invention, the second agent can be or comprise any agent that acts in a manner other than as an inhibitor of Lp-PLPA2. By way of example, and not to be construed as limiting the scope of the invention, the second agent can be or comprise an amyloid-targeting molecule (such as, but not limited to a molecule targeting Aβ amyloid proteins), a tau-targeting molecule, a vasculature-targeting molecule, an inflammation-targeting molecules, or a molecule that modulates a mechanism of action that is otherwise orthogonal from Lp-PLA2 inhibition (such as, but not limited to, agents having mechanisms of action other than those listed above, e.g., including epigenetic mechanism-targeting therapies, metabolism-targeting therapies, bioenergetics-targeting therapies, neurogenesis-targeting therapies, neurotransmitter-targeting therapies, neurotransmitter receptor-targeting therapies, oxidative stress-targeting therapies, proteostasis-targeting therapies, synaptic plasticity-targeting therapies, neuroprotection-targeting therapies, and the like). Several compounds suitable for use as “second agents” also are undergoing clinical investigation. See, for example, Cummings et al. Alzheimer's & Dementia: Translational Research & Clinical Interventions 2022; doi.org/10.1002/trc2.12295, which is incorporated herein its entirety by reference. Of course, the invention is not limited to these agents. Moreover, combinations of these agents, as well as the inclusion of agents that act through yet other mechanisms, are expressly contemplated for use in the context of the present invention.

By way of non-limiting examples, for instance, when the second agent comprises an amyloid-targeting molecule, the second agent can be one or more agents such as ABvac40, ACU193, Aducanumab (ADUHELM), APH-1105, Brain Shuttle Gantenerumab (RO7126209), Crenezumab, Donanemab (LY3002813), Gantenerumab, Lecanemab (BAN2401, LEQEMBI), LY3372993, MIB-626, SHR-1707, Solanezumab, Thiethylperazine (TEP), Valiltramiprosate (ALZ-801), Varoglutamstat (PQ912), and the like. Also, more than one of such (and/or other) amyloid-targeting molecules can be employed as the “second agent” as well. Several non-limiting examples of amyloid inhibitors suitable for use as the “second agent(s)” in the context of the present invention (i.e., in combination with the first agent targeting Lp-PLA2) either have been approved or are undergoing investigational clinical trials at various stages. The following table (Table 1) lists some examples of these, which approaches can be combined with a first agent targeting Lp-PLA2 in the context of the present invention.

TABLE 1

| Compound | Clinical stage as of April 2023 |
|---|---|
| ACU193 | Phase 1 |
| LY3372993 | |
| SHR-1707 | |
| ABvac40 | Phase 2 |
| APH-1105 | |
| Valiltramiprosate (ALZ-801) | |
| Varoglutamstat (PQ912) | |
| Thiethylperazine (TEP) | |
| Lecanemab (BAN2401) | |
| Brain Shuttle Gantenerumab | |
| (RO7126209) | |
| MIB-626 | |
| Gantenerumab | |
| Donanemab (LY3002813 | |
| Crenezumab | |
| Donanemab | Phase 3 |
| Donanemab & Aducanumab | |
| Gantenerumab & Solanezumab | |
| Gantenerumab | |
| Lecanemab (BAN2401)/Leqembi | |
| Valiltramiprosate (ALZ-801) | |
| Solanezumab | |
| Aducanumab/Aduhelm | |

Total molecules in Phase 1-Phase 3: 22

Furthermore, also by way of non-limiting example, for instance, when the second agent comprises a tau-targeting molecule, the second agent can be one or more agents such as ACI-35, ASN51, Bepranemab, BIIB080/IONIS-MAPTRx, E2814, IONIS MAPTRx (BIIB080), JNJ-63733657, Lu AF87908, LY3372689, Nicotinamide, PU-AD, Semorinemab (RO7105705), TB006, and the like. More than one of such (and/or other) tau-targeting molecules can be employed as the “second agent” as well. Several non-limiting examples of tau-targeting molecules suitable for use as the “second agent(s)” in the context of the present invention (i.e., in combination with the first agent targeting Lp-PLA2) either have been approved or are undergoing investigational clinical trials at various stages. The following table (Table 2) lists some examples of these, which approaches can be combined with a first agent targeting Lp-PLA2 in the context of the present invention.

TABLE 2

| Compound | Clinical stage as of April 2023 |
|---|---|
| ASN51 | Phase 1 |
| Lu AF87908 | |
| TB006 | |
| BIIB080/IONIS-MAPTRx | |
| JNJ-63733657 | Phase 2 |
| LY3372689 | |
| Nicotinamide | |
| PU-AD | |
| Semorinemab (RO7105705) | |
| ACI-35 | |
| Bepranemab | |
| IONIS MAPTRx (BIIB080) | |
| E2814 | |
| TRx0237 | Phase 3 |

Total molecules in Phase 1-Phase 3: 14

Furthermore, also by way of non-limiting examples, for instance, when the second agent comprises a vasculature-targeting molecule, the second agent can be one or more agents such as Amlodipine, AR1001, Atorvastatin, Dabisatran, Losartan, Omega-3 PUFA, Perindorpil, Renew NCP5, Telmisartan, Yangxue Qingnao pills, and the like. More than one of such (and/or other) vasculature-targeting molecules can be employed as the “second agent” as well. Indeed, it is contemplated that, in some embodiments, the invention will involve the use of two or more vascular-targeting molecules. Such may increase efficacy, such as by targeting separate vasculature pathways simultaneously (e.g. BBB leakage/integrity plus vascular inflammation, as a non-limiting example). Several non-limiting examples of vasculature-targeting molecules suitable for use as the “second agent(s)” in the context of the present invention (i.e., in combination with the first agent targeting Lp-PLA2) either have been approved or are undergoing investigational clinical trials at various stages. The following table (Table 3) lists some examples of these, which approaches can be combined with a first agent targeting Lp-PLA2 in the context of the present invention.

TABLE 3

| Compound | Clinical stage as of April 2023 |
|---|---|
| Dabisatran | Phase 1 |
| AR1001 | Phase 2 |
| Perindorpil/Telmisartan | |
| Omega-3 PUFA | |
| Yangxue Qingnao pills | |
| Renew NCP5 | Phase 3 |
| Losartan & Amlodipine & Atorvastatin + | |
| exercise | |

Total molecules in Phase 1-Phase 3: 7

Furthermore, also by way of non-limiting examples, for instance, when the second agent comprises an inflammation-targeting molecule, the second agent can be one or more agents such as AL002, Baricitinib, BCG vaccine, Canakinumab, Curcumin, Daratumumab, Dasatinib, Edicotinib (JNJ-40346527), Emtricitabine, GB301, L-Serine, Lenalidomide, Montelukast, NE3107, Pepinemab (VX15), Quercetin, Salsalate, Sargramostim, Senicapoc, TB006, Tdap vaccine, Valacyclovir, VT301, XPro1595, and the like. More than one of such (and/or other) inflammation-targeting molecules can be employed as the “second agent” as well. Several non-limiting examples of inflammation-targeting molecules suitable for use as the “second agent(s)” in the context of the present invention (i.e., in combination with the first agent targeting Lp-PLA2) either have been approved or are undergoing investigational clinical trials at various stages. The following table (Table 4) lists some examples of these, which approaches can be combined with a first agent targeting Lp-PLA2 in the context of the present invention.

TABLE 4

| Compound | Clinical stage as of April 2023 |
|---|---|
| Edicotinib (JNJ-40346527) | Phase 1 |
| Emtricitabine | |
| Salsalate | |
| VT301 | |
| XPro1595 | |
| AL002 | Phase 2 |
| Baricitinib | |
| BCG vaccine | |
| Canakinumab | |
| Curcumin + aerobic yoga | |
| Daratumumab | |
| Dasatinib + Quercetin | |
| GB301 | |
| Lenalidomide | |
| L-Serine | |
| Montelukast | |
| Pepinemab (VX15) | |
| Sargramostim | |
| Senicapoc | |
| TB006 | |
| Tdap vaccine | |
| Valacyclovir | |
| NE3107 | Phase 3 |

Total molecules in Phase 1-Phase 3: 23

Furthermore, also by way of non-limiting examples, for instance, when the second agent comprises an agent having a mechanism of action that is orthogonal from Lp-PLA2 inhibition, the second agent can be one or more agents such as AAV-Htert, AD-35, AGB101 (low-dose levetiracetam), Allopregnanolone, Atuzaginstat (COR388), AVP-786, AXS-05, BDPP (bioactive dietary polyphenol preparation), BEY2153, Blarcamesine (ANAVEX2-73), BMS-984923, BPDO-1603, BPN14770, Brexpiprazole, Bromocriptine, Bryostatin 1, BXCL-501, Caffeine, Contraloid acetate, COR588, CORT108297, CST-2032, CY6463, DAOIB, Dapagliflozin, Deferiprone, DHA, Donepezil, Dronabinol, Edonerpic (T-817MA), Efavirenz, Elayta (CT1812), Escitalopram, ExPlas (exercised plasma), Fosgonimeton (ATH-1017), GC AD1, Grapeseed extract, Guanfacine, GV-971, GV1001, Hydralazine, Icosapent ethyl (IPE), imufilam (PTI-125), Intranasal insulin, Intranasal insulin+Empagliflozin, Lamivudine (3TC), Levetiracetam, Lupron (leuprolide acetate depot), LX1001, Memantine, Metabolic cofactor supplementation, Metformin, MK-1942+donepezil, MW150, Nabilone, Neflamapimod (VX-745), Nicotine transdermal patch, Nilotinib BE, NNI-362, Obicetrapib, Octohydro-aminoacridine Succinate, Omega-3 (DHA+EPA), Posiphen, Prazosin, Rapamycin (sirolimus), REM0046127, Semaglutide, Simufilam (PTI-125), Sovateltide (PMZ-1620), Suvorexant, T3D-959, THC-free CBD oil, Trehalose, Tricaprilin, Tricaprilin (AC-1202), Troriluzole (BHV4157), Xanamem, and the like. The following table (Table 5) lists some examples of these, which approaches can be combined with a first agent targeting Lp-PLA2 in the context of the present invention.

TABLE 5

| | Mechanism of action orthogonal | Current clinical stage |
|---|---|---|
| Compound | to Lp-PLA2 inhibition | as of April 2023 |
| AAV-Htert | Epigenetic | P1 |
| Allopregnanolone | Neurogenesis | |
| BEY2153 | Proteostasis | |
| BMS-984923 | Synaptic plasticity/neuroprotection | |
| BDPP (bioactive dietary | Proteostasis | |
| polyphenol preparation) | | |
| Contraloid acetate | Proteostasis | |
| COR588 | Synaptic plasticity/neuroprote | |
| Donepezil | Neurotransmitter receptors | |
| Efavirenz | Epigenetics | |
| GC AD1 | Neurotransmitter receptors | |
| LX1001 | Epigenetic | |
| MK-1942 + donepezil | Neurotransmitter receptors | |
| NNI-362 | Neurogenesis | |
| REM0046127 | Synaptic Plasticity/Neuroprotection | |
| Trehalose | Cell death | |
| Tricaprilin (AC-1202) | Metabolism and bioenergetics | |
| AD-35 | Neurotransmitter receptors | P2 |
| Allopregnanolone | Neurogenesis | |
| BPN14770 | Synaptic plasticity/neuroprotection | |
| Bromocriptine | Neurotransmitter receptors | |
| Bryostatin 1 | Synaptic plasticity/neuroprotection | |
| BXCL-501 | Neurotransmitter receptors | |
| CORT108297 | Hormones | |
| CST-2032 | Neurotransmitter receptors | |
| CY6463 | Synaptic Plasticity/Neuroprotection | |
| DAOIB | Neurotransmitter receptors | |
| Dapagliflozin | Metabolism and bioenergetics | |
| Deferiprone | Cell death | |
| DHA | Oxidative stress | |
| Dronabinol | Neurotransmitter receptors | |
| Edonerpic (T-817MA) | Synaptic plasticity/neuroprotection | |
| Elayta (CT1812) | Synaptic plasticity/neuroprotection | |
| ExPlas (exercised plasma) | Synaptic plasticity/neuroprotection | |
| Fosgonimeton (ATH-1017) | Synaptic plasticity/neuroprotection | |
| Grapeseed extract | Proteostasis | |
| GV1001 | Epigenetic | |
| Intranasal insulin | Metabolism and bioenergetics | |
| Intranasal insulin + | Metabolism and bioenergetics | |
| Empagliflozin | | |
| Lamivudine (3TC) | Epigenetic | |
| Levetiracetam | Synaptic plasticity/neuroprotection | |
| Lupron (leuprolide acetate | Growth factors and hormones | |
| depot) | | |
| Memantine | Neurotransmitter receptors | |
| Metabolic cofactor | Metabolism and bioenergetics | |
| supplementation | | |
| MW150 | Synaptic plasticity/neuroprotection | |
| Neflamapimod (VX-745) | Synaptic plasticity/neuroprotection | |
| Nicotine transdermal patch | Neurotransmitter receptors | |
| Obicetrapib | Lipids and lipoprotein receptors | |
| Posiphen | Proteostasis | |
| Prazosin | Neurotransmitter receptors | |
| Rapamycin (sirolimus) | Proteostasis | |
| Sovateltide (PMZ-1620) | Neurogenesis | |
| imufilam (PTI-125) | Synaptic plasticity/neuroprotection | |
| Suvorexant | Neurotransmitter receptors | |
| T3D-959 | Metabolism and bioenergetics | |
| THC-free CBD oil | Neurotransmitter receptors | |
| Troriluzole (BHV4157) | Synaptic plasticity/neuroprotection | |
| Xanamem | Growth factors and hormones | |
| AGB101 (low-dose | Synaptic Plasticity/Neuroprotection | P3 |
| levetiracetam) | | |
| Atuzaginstat (COR388) | Synaptic Plasticity/Neuroprotection | |
| AVP-786 | Neurotransmitter receptors | |
| AXS-05 | Neurotransmitter receptors | |
| Blarcamesine | Synaptic plasticity/neuroprotection | |
| (ANAVEX2-73) | | |
| BPDO-1603 | Undisclosed | |
| Brexpiprazole | Neurotransmitter receptors | |
| Caffeine | Neurotransmitter receptors | |
| Donepezil | Neurotransmitter receptors | |
| Escitalopram | Neurotransmitter receptors | |
| Guanfacine | Neurotransmitter receptors | |
| GV-971 | Gut-brain axis | |
| Hydralazine | Oxidative stress | |
| Icosapent ethyl (IPE) | Oxidative stress | |
| Metformin | Metabolism and bioenergetics | |
| Nabilone | Neurotransmitter receptors | |
| Nilotinib BE | Proteostasis | |
| Octohydro-aminoacridine | Neurotransmitter receptors | |
| Succinate | | |
| Omega-3 (DHA + EPA) | Oxidative stress | |
| Semaglutide | Metabolism and bioenergetics | |
| Simufilam (PTI-125) | Synaptic Plasticity/Neuroprotection | |
| Tricaprilin | Metabolism and bioenergetics | |

Total molecules in P1-3: 79

It will be observed that the first agent (Lp-PLA2-inhibitor) and the second agent can be formulated together in a common composition or administered separately, as desired. When administered separately, the first agent and the second agent can be administered simultaneously or consecutively. By “simultaneously,” it is meant that the first and second agents are administered together contemporaneously, whether in the same or separate formulations. By “consecutively” it is meant that the administration of either the first agent or the second agent precedes the administration of the other. Moreover, the administration of the first agent and the second agent can be such that one administration is begun before the other, and/or one administration is ended before the other, but there is a period of overlap during which the administration is simultaneous. In some embodiments, the first agent is administered before the second agent (such as repeatedly administered before the second agent), which can establish a favorable condition for the administration of the second agent. For example, the first agent can exert a protective effect against vascular damage and/or inflammation that may otherwise be caused by the administration of the second agent, if the second agent were administered either as monotherapy or concurrently with the first agent. Moreover, either or both agents can be administered simultaneously or sequentially (repeatedly) one or several times (e.g., at least 2, or at least 3, or at least 4 times or more) either concurrently or prior to the administration of the other.

Moreover, the inventive combination therapy can be repeated over a period of time (e.g., once, twice, three, four, etc. times daily for one or several days, or weeks, or months (e.g., for about 24 or more weeks). The amount and dosage regimen will necessarily vary in accordance with the mode and route of administration, as well as with the identity of the first and second agent. However, it is within the skill of the art to select an appropriate dose and route of administration to deliver the first and second agents to the subject in accordance with the inventive method.

As many of the agents suitable for use in the context of the invention are approved medications or are undergoing human clinical trials (see, by way of non-limiting examples, the tables above), in many embodiments, the extant formulations, dosages, and regiments approved or under investigation for such agents can be employed in the context of the present invention as well. For example, as noted, where rilapladib is employed as the first agent, it can be administered as per the regimen employed in clinical trials (dosages of from 2.5-400 mg/day for up to 24 weeks, such as 250 mg rilapladib once daily (see clinical trial identifiers NCT01750827, NCT00387257, NCT01745458, and NCT01428453) or as otherwise deemed appropriate by a treating physician. In this respect, as noted above, it has been reported that rilapladib can achieve approximately 80% inhibition of Lp-PLA2 when at a dosage of 250 mg to human subjects. However, the invention also provides a pharmaceutical composition comprising a first agent and a second agent, as described herein, and a pharmaceutically-acceptable excipient.

For use in the context of the present invention, the inventive composition can be formulated for any desired mode and route of administration, such as orally, transdermally, interperitoneally, intravenously, intracerebrally, or otherwise as deemed appropriate for a particular subject or patient by the treating physician. Accordingly, the inventive composition can comprise, as components of the pharmaceutically-acceptable carrier, various adjuvants and excipients as are typically employed by pharmaceutical formulation methodology. Thus, for example, for injection (such as, but not limited to, intravenous or intraperitoneal injection), the formulation can be or comprise an aqueous solution or suspension comprising saline and other desired components. The composition also can contain preservatives, other active or inactive agents, stability enhancers, and the like. Moreover, the inventive composition can be filed into vials, compressed into tablets, filled into capsules, or otherwise packaged for unit-dose formulation or for repeat administration, as desired.

It will be observed that the inventive method involving combination therapy, and the associated inventive composition, can be used in the treatment and/or prophylaxis of a variety of conditions, diseases and disorders. Among these are included those involving vascular damage and/or inflammation, particularly (but not limited to) those associated with neurological or neuromuscular conditions. Such diseases can be defined clinically in the sense that they need not (but may) present as symptoms in a patient or subject. For example, Alzheimer's Disease (AD) can be identified in a living subject by assaying the Aβ42/40 ratio in CSF or plasma, or pTau181, or pTau217 levels in CSF or plasma (diagnostic) and need not depend on the presentation of discernable symptoms. In this respect, certain published diagnostic criteria for AD include (See Media & Investor Release, “Roche Alzheimer's disease Cerebrospinal Fluid (CSF) assays receive FDA clearance, supporting more accurate and timely diagnosis” (Dec. 8, 2022) and “The Power of Elecsys® in Alzheimer's disease” (Roche), which are incorporated herein in their entireties):
If Aβ42≤1,000 pg/mL test result positive.
If Aβ42>1,000 pg/mL test result negative.
If pTau/Aβ42 ratio>0.024 test result positive.
If pTau/Aβ42 ratio≤0.024 test result negative.
If tTau/Aβ42 ratio*>0.28 test result positive.
If tTau/Aβ42 ratio*≤0.28 test result negative
   Similarly, these published diagnostic criteria also contain thresholds for identifying patients at risk of cognitive decline:
If pTau/Aβ42 ratio>0.024 test result positive.
If pTau/Aβ42 ratio≤0.024 test result negative.
If tTau/Aβ42 ratio*>0.28 test result positive.
If tTau/Aβ42 ratio*≤0.28 test result negative.

While the inventive use of combination therapy, including the aforementioned method and associated composition, can be employed as prophylaxis or treatment of a variety of conditions, diseases and disorders, it is contemplated the invention will be particularly useful in the context of conditions, diseases and disorders associated with age-related diseases, diabetes, mental disorders, NDDs, VCIDs, CSVDs, neurological injuries or diseases, proteinopathies, tauopathies, vascular diseases, and conditions, diseases and disorders involving combinations of one or more of these. For instance, in certain embodiments, non-limiting examples of conditions, diseases and disorders associated with age-related disease that can be addressed by the inventive use, method, and composition include, but are not limited to hypertension, heart disease, congestive heart failure, high blood pressure, coronary artery disease, coronary heart disease, dementia, Alzheimer's disease, Lewy body dementia, vascular dementia, frontotemporal dementia, delirium, depression, anxiety, epilepsy, high cholesterol, hypertension, motor neuron disease, multiple sclerosis, osteoporosis, Paget's disease of bone, shingles, stroke, incontinence (urinary and bowel), urinary tract infections (UTIs), chronic kidney disease, deep vein thrombosis, arthritis, osteoporosis, diabetes, lung disease, asthma, chronic obstructive pulmonary disease, bronchitis, pneumonia, influenza, frequent falls, which can lead to bone fractures, Parkinson's disease, sleep disorders, insomnia, sleep apnea, restless leg syndrome, cancer, eye diseases, blindness, cataracts, glaucoma, macular degeneration, dry eye, low vision, weight loss, and combinations of one or more of these.

Furthermore, in certain embodiments, non-limiting examples of conditions, diseases and disorders associated with mental disorders that can be addressed by the inventive use, method, and composition include, but are not limited to autism, ADHD, dyslexia, Tourette's syndrome, schizophrenia, bipolar disorder, clinical depression, panic disorder, phobias, generalized anxiety disorder, obsessive-compulsive disorder (OCD), post-traumatic stress disorder (PTSD), depersonalization disorder, dissociative identity disorder, illness anxiety disorder, conversion disorder, anorexia nervosa, bulimia nervosa, narcolepsy, oppositional defiant disorder, conduct disorder, and combinations of one or more of these. Similarly, in certain embodiments, non-limiting examples of conditions, diseases and disorders associated with NDDs that can be addressed by the inventive use, method, and composition include, but are not limited to Alzheimer's disease, amyotrophic lateral sclerosis, Huntington's disease, Lewy body dementia, multiple sclerosis, multiple system atrophy, Parkinson's disease, prion disease, HIV and Dementia, and combinations of one or more of these.

Furthermore, in certain embodiments, non-limiting examples of conditions, diseases and disorders associated with mental disorders that can be addressed by the inventive use, method, and composition include central nerve disorders, peripheral nerve disorders, and neuromuscular disorders. Examples of such, which are presented to illustrate the scope of the invention and not to limit it, include, for instance, brachial neuritis, carpal tunnel syndrome, diabetic neuropathy, meralgia paresthetica, neurofibroma, peripheral neuropathies, sciatica, tardive dyskinesia, thoracic outlet syndrome, ulnar nerve entrapment, pain disorders, causalgia, complex regional pain syndrome, glossopharyngeal neuralgia, phantom limb pain, postherapeutic neuralgia, trigeminal neuralgia, ALS, amyotrophic lateral sclerosis (Lou Gehrig's disease), ataxia, brachial neuritis, carnitine palmitoyl transferase deficiency, carpal tunnel syndrome, Charcot-Marie-tooth, chronic inflammatory demyelinating, polyradiculoneuropathy, diabetic neuropathy, Friedreich's ataxia, Guillain-Barre syndrome, hemiballismus, Lambert-Eaton syndrome, McArdle disease, multiple system atrophy (MSA), muscular dystrophy, distal muscular dystrophy, Duchenne muscular dystrophy, oculopharyngeal muscular dystrophy (OPMD), myasthenia gravis, myopathy, myositis, neuromuscular disorders, polymyositis, polymyositis and dermatomyositis, postherapeutic neuralgia, post-polio syndrome, spinal muscular atrophy, multiple sclerosis, cerebellar tremor, clinically isolated syndrome (cis), connective tissue disorders, multiple sclerosis (MS), fulminate MS, MS and pregnancy, neuromyelitis optica (NMO) (Devic's disease), neurosarcoidosis, primary-progressive MS (PPMS), progressing-relapsing MS (PRMS), radiologically isolated syndrome (RIS), relapsing-remitting MS (RRMS), secondary-progressive MS (SPMS), transverse myelitis, tumefactive MS, akathisia, ataxia, athetosis, atypical parkinsonism, ballism, blepharospasm, cerebellar tremor, chorea, corticobasal degeneration (CBD), dyskinesia, dystonia, dystonic tremor, Friedreich's ataxia, Emery-Dreyfuss muscular dystrophy in children, essential tremor, focal dystonia, motor and vocal tics. Parkinson's disease & dementia, paroxysmal dyskinesia, progressive supranuclear palsy (PSP), restless legs syndrome (RLS), Sydenham's chorea, spasmodic torticollis, spasticity, tardive dyskinesia, tic disorder, tremor, torticollis, Tourette syndrome, Wilson's disease, writer's cramp, epilepsy & seizures, absence seizures, akinetic seizures, atonic seizures, atypical absence seizures, auras, benign Rolandic epilepsy of childhood, catamenial epilepsy, cerebellar tremor, complex partial seizures, drop attacks, dystonic tremor, epilepsia partialis continua, epilepsy, epilepsy and seizures, epilepsy caused by tumors or malformations of blood vessels, epilepsy of Janz, essential tremor, focal seizures, fugue states, gelastic seizures, generalized seizures, grand mal seizures, infantile spasms, Jacksonian seizures, juvenile myoclonic epilepsy, Landau-Kleffner syndrome, Lennox-Gastaut syndrome, menstrual seizures, mesial frontal lobe epilepsy, mesial temporal lobe epilepsy, motor seizures, myoclonic epilepsy, myoclonus, nocturnal seizures, other extratemporal lobe epilepsies, parasomnia epilepsy, partial seizures, petit mal seizures, photosensitive epilepsy, physiological tremor, post-traumatic seizures, rage attacks, Rasmussen's encephalitis, reading epilepsy, refractory seizures, sensory seizures, simple partial seizures, subclinical seizures, status epilepticus. headache, cluster headaches, headache, migraine headaches, tension headaches, trigeminal neuralgia, chemobrain, and combinations of one or more of these.

Furthermore, in certain embodiments, non-limiting examples of conditions, diseases and disorders associated with tauopathies that can be addressed by the inventive use, method, and composition include, but are not limited to primary age-related tauopathy, chronic traumatic encephalopathy, progressive supranuclear palsy, corticobasal degeneration, frontotemporal dementia and parkinsonism linked to chromosome 17, vacuolar tauopathy, lytico-bodig disease, ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, behavioral variant frontotemporal dementia (Pick's disease), argyrophilic grain disease, and combinations of one or more of these.

Furthermore, in certain embodiments, non-limiting examples of conditions, diseases and disorders associated with vascular diseases that can be addressed by the inventive use, method, and composition include, but are not limited to cerebrovascular disease, cerebral small vessel disease, blood-brain barrier leakage, atherosclerosis, cerebral amyloid angiopathy, peripheral vascular disease, peripheral artery disease carotid artery disease, pulmonary embolism, abdominal aortic aneurism, collagen vascular disease, chronic venous insufficiency thrombosis, deep vein thrombosis, acute subdural hematoma, arterial cranio-cervical trauma, arterial venous fistula: cerebral or spinal, arteriovenous malformation (AVM), brain aneurysm (cerebral aneurysms), carotid dissection, carotid artery stenosis, cavernous angioma, cavernous malformation, cerebral arteriovenous malformations, cerebral contusion and intracerebral hematoma, extracranial-intracranial bypass, fibromuscular dysplasia, intracranial arterial stenosis, Moya Moya disease, non-ruptured cerebral aneurysm, spasticity, spina bifida in children, spinal arteriovenous malformation (AVM), subarachnoid hemorrhage, subdural hematoma, venous sinus thrombosis, vertebral artery dissection, vertebral artery stenosis, vertebral osteomyelitis, vascular diseases with CSF leakage including cerebrospinal fluid diverting shunts, cerebrospinal fluid leak, intracranial hypotension, normal pressure hydrocephalus, pseudotumor cerebri, traumatic brain injury, and combinations of one or more of these.

Furthermore, in certain embodiments, non-limiting examples of conditions, diseases and disorders associated with proteinopathies that can be addressed by the inventive use, method, and composition include, but are not limited to Creutzfeldt-Jakob disease, prion diseases, Alzheimer's disease, Parkinson's disease, tauopathies, α-synucleinopathies, dementia with lewy bodies, amyloidosis, multiple system atrophy, frontotemporal dementia with tau inclusions, frontotemporal dementia with TDP-43 inclusions, frontotemporal dementia with FUS inclusions, amyotrophic lateral sclerosis with SOD1 inclusions, amyotrophic lateral sclerosis with FUS inclusions, amyotrophic lateral sclerosis with TDP-43 inclusions, Huntington disease, Progressive supranuclear palsy, Posterior cortical atrophy, and combinations of one or more of these.

The inventive use of combination therapy and prophylaxis and associated method and composition can be employed advantageously in several respects. For example, in certain embodiments, the invention can beneficially mitigate vascular damage, mitigate vascular inflammation, improve the safety of, improve the therapeutic index of, improve efficacy of existing monotherapies. The invention also has an advantage of expanding the potential patient population eligible for certain therapies.

By way of an example, currently, therapies targeting amyloid proteins (such as Aβ-targeting therapies, such as monoclonal antibodies) hold promise for the treatment of AD. However, in certain patients, such agents targeting amyloid proteins cause clinical side effects. For example, Aβ-targeting therapies, such as monoclonal antibodies, have been shown to reduce Aβ levels in the brain, but they have also been associated with treatment related cerebral microbleeds and vascular damage (such as ARIA-E and ARIA-H). Currently, when patients experience such side-effects, the clinical response is to discontinue therapy. Thus, given the vascular protection afforded by targeting Lp-PLA2, the invention, involving the use of agents inhibiting Lp-PLA2 together with such therapies targeting amyloids, can mitigate the detrimental vascular events, thereby improving the safety, tolerability of amyloid-targeting therapy for such patients. The vascular damage repairment contributed by Lp-PLA2 inhibition could, for example, help alleviate ARIA-E and/or ARIA-H side effects of anti-amyloid antibody therapies and provide better efficacy to patients, especially those carrying one (heterozygous) or two (homozygous) apolipoprotein E epsilon 4 (APOE-84 or APOE4) allele of the APOE gene, by optimizing dosing regimens. Moreover, the patient population suitable for anti-amyloid therapy can thereby be increased as discontinuation of such therapy may not be necessary when administered in combination with Lp-PLA2-inihibitors in accordance with the invention.

In a similar vein, many elderly patients, as well as patients with advanced stage AD, or presenting with vascular co-morbidities or a history of cerebral hemorrhage and microbleeds currently have restricted or no access to potential life-saving amyloid-targeting therapies. However, given the vascular protection afforded by targeting Lp-PLA2, the invention, involving the use of agents inhibiting Lp-PLA2 together with such therapies targeting amyloids, can mitigate the disease-related vascular events, thereby improving the safety, tolerability of amyloid-targeting therapy for this population of AD patients for which current therapies are contraindicated. Moreover, the AD patient population suitable for anti-amyloid therapy can thereby be increased.

As another non-limiting example, the invention can usefully improve the potential for current or investigational CNS disease modification monotherapy. The pathologic mechanisms that are driving NDD onset, progression and severity are multiple and often poorly understood. Accordingly, the treatment of such polygenic and multifactorial diseases may require the normalization of more than one biological pathway. This is consistent with the low success rate of monotherapies in treating neurological diseases and exemplifies the urgent need to provide more potent therapeutic approaches. As non-limiting examples, Lp-PLA2 inhibitors used in combination with other drugs targeting orthogonal mechanisms for the treatment of NDD, proteinopathies is contemplated. For instance, combining Lp-PLA2 inhibition benefiting vascular aspects of CNS disease pathology with another agent that targeting, for example, the accumulation of a pathogenic protein such as tau, alpha-synuclein and TDP43 is contemplated.

It will be understood that, in the context of the present invention, the subject to be treated is typically human (i.e., a patient), but the subject can be a non-human animal instead, typically mammalian (such as a pet animal, livestock, or animal typically employed in laboratory research, such as cats, dogs, horses, cows, mice, rats, and rabbits). When the patient is human, the patient can be contraindicated for monotherapies, such as, for example, carrying the APOE4 allele or presenting with cerebral amyloid angiopathy (CAA).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of therapeutic or prophylactic treatment of vascular damage and/or inflammation associated with a disease, condition, or therapy within a subject comprising administering a first agent to the subject, wherein the first agent comprises a lipoprotein-associated phospholipase A2 (Lp-PLA2)-inhibitor molecule, and administering a second agent to the subject, wherein the second agent comprises an amyloid-targeting molecule, an agent that lowers the level of amyloid in the brain or body, a tau-targeting molecule, a vasculature-targeting molecule, an inflammation-targeting molecule, a molecule that modulates a mechanism of action that is orthogonal from Lp-PLA2 inhibition, or a combination thereof, whereby the combined administration of the first and the second agent effects therapeutic prophylaxis or treatment of vascular damage and/or inflammation within the subject.

2-4. (canceled)

5. The method of claim 1, wherein the first agent comprises darapladib, rilapladib, GSK3206906A (SNP318), a fragment and derivative, modified format, or mimetic of darapladib, rilapladib, or GSK3206906A (SNP318) that inhibits Lp-PLA2, or a combination thereof.

6. (canceled)

7. The method of claim 1, wherein the second agent comprises an amyloid-targeting molecule.

8. (canceled)

9. The method of claim 1, wherein the second agent comprises an agent that lowers the level of amyloid in the brain or body.

10. (canceled)

11. The method of claim 1, wherein the second agent comprises a tau-targeting molecule.

12. (canceled)

13. The method of claim 1, wherein the second agent comprises a vasculature-targeting molecule.

14. (canceled)

15. The method of claim 1, wherein the second agent comprises an inflammation-targeting molecule.

16. (canceled)

17. The method of claim 1, wherein the second agent comprises a molecule that modulates a mechanism of action that is orthogonal from Lp-PLA2 inhibition.

18. (canceled)

19. The method of claim 1, wherein the vascular damage or inflammation is associated with a disease or disorder comprising an age-related disease, diabetes, a mental disorder, a neurodegenerative disease (NDD), vascular cognitive impairment and dementia (VCID), Cerebral small vessel disease (CSVD), a neurological injury or disease, a proteinopathy, a tauopathy, a vascular disease, treatment-related vascular damage or inflammation, or a combination thereof.

20-35. (canceled)

36. The method of claim 19, wherein the treatment-related vascular damage or inflammation is attributable to the second agent when the second agent is employed as a monotherapy

37-44. (canceled)

45. The method of claim 1, wherein the first agent and/or the second agent are administered repeatedly.

46. The method of claim 1, wherein the subject is a human patient.

47-48. (canceled)

49. A composition comprising a first agent comprising a lipoprotein-associated phospholipase A2 (Lp-PLA2)-inhibitor molecule, a second agent comprising an amyloid-targeting molecule, an agent that lowers the level of amyloid in the brain or body, a tau-targeting molecule, a vasculature-targeting molecule, an inflammation-targeting molecule, a molecule that modulates a mechanism of action that is orthogonal from Lp-PLA2 inhibition, or a combination thereof, and a pharmaceutical carrier.

50. The composition of claim 49, wherein the first agent comprises darapladib, rilapladib, GSK3206906A (SNP318), a fragment and derivative, modified format, or mimetic of darapladib, rilapladib, or GSK3206906A (SNP318) that inhibits Lp-PLA2, or a combination thereof.

51-52. (canceled)

53. The composition of claim 49, wherein the second agent comprises an amyloid-targeting molecule.

54. (canceled)

55. The composition of claim 49, wherein the second agent comprises an agent that lowers the level of amyloid in the brain or body.

56. (canceled)

57. The composition of claim 49, wherein the second agent comprises a tau-targeting molecule.

58. (canceled)

59. The composition of claim 49, wherein the second agent comprises a vasculature-targeting molecule.

60. (canceled)

61. The composition of claim 49, wherein the second agent comprises an inflammation-targeting molecule.

62. (canceled)

63. The composition of claim 49, wherein the second agent comprises a molecule that modulates a mechanism of action that is orthogonal from Lp-PLA2 inhibition.

64-65. (canceled)
